(19) United States
(12) Patent Application Publication
(10) Pub. No.: US 2023/0377702 A1
(43) Pub. Date: Nov. 23, 2023

(54) MEDICAL INFORMATION PROCESSING APPARATUS AND CONSISTENCY DETERMINATION METHOD

(71) Applicant: CANON MEDICAL SYSTEMS CORPORATION, Otawara-shi (JP)

(72) Inventors: Hayato OKUMIYA, Nasushiobara (JP); Atsuko SUGIYAMA, Nasushiobara (JP)

(73) Assignee: CANON MEDICAL SYSTEMS CORPORATION, Otawara-shi (JP)

(21) Appl. No.: 18/319,537

(22) Filed: May 18, 2023

(30) Foreign Application Priority Data
May 20, 2022 (JP) 2022-083290

(51) Int. Cl. G16H 10/60; G16H 50/70

(52) U.S. Cl. CPC G16H 10/60; G16H 50/70

(57) ABSTRACT

A medical information processing apparatus according to an embodiment includes processing circuitry. The processing circuitry configured to acquire a first character string from medical treatment information, acquire one or more second character strings estimated from the first character string, from at least one of the medical treatment information and one or more pieces of medical information that is different from the medical treatment information, determine whether a content indicated by the first character string is consistent with a content indicated by the second character string, and output specific information for specifying a location of the first character string, if the content indicated by the first character string is inconsistent with the content indicated by the second character string.

### CROSS-REFERENCE TO RELATED APPLICATIONS

This application is based upon and claims the benefit of priority from Japanese Patent Application No. 2022-083290, filed on May 20, 2022, the entire contents of which are incorporated herein by reference.

### FIELD

Embodiments described herein relate generally to a medical information processing apparatus and a consistency determination method.

### BACKGROUND

Conventionally, health care professionals enter the progress of medical treatment and the like into electronic medical records. The health care professionals may enter errors due to input mistakes, misunderstandings, or the like. Therefore, a health care professional different from the health care professional who has entered the information checks whether the electronic medical records contain errors.

However, it is time consuming to manually check whether the medical information such as electronic medical records contain errors.

### BRIEF DESCRIPTION OF THE DRAWINGS

FIG. 1 is a block diagram illustrating an example of a configuration of a consistency checking system according to a first embodiment;

FIG. 2 is a flowchart illustrating an example of a consistency determination process executed by a medical information processing apparatus according to the first embodiment;

FIG. 3 is a block diagram illustrating an example of a configuration of a consistency checking system according to a second embodiment;

FIG. 4 is a diagram illustrating an example of an input candidate image;

FIG. 5 is a diagram illustrating an example of a save confirmation image; and

FIG. 6 is a flowchart illustrating an example of an input support process executed by a medical information processing apparatus according to the second embodiment.

### DETAILED DESCRIPTION

Hereinafter, a medical information processing apparatus, a consistency determination method, and an input candidate display method according to the present embodiment will be described in detail with reference to the accompanying drawings. In the following embodiments, the parts with the same reference numerals perform the same operation, and duplicate description will be omitted as appropriate.

### First Embodiment

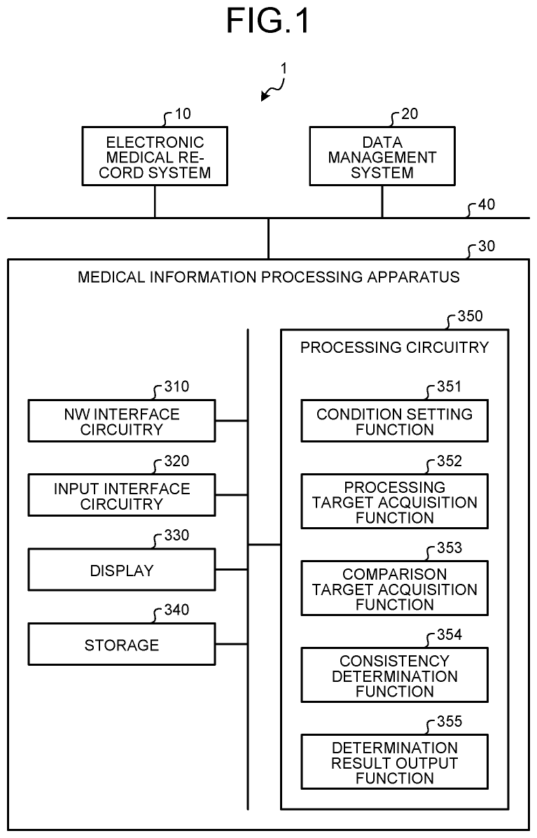

FIG. 1 is a block diagram illustrating an example of a configuration of a consistency checking system 1 according to a first embodiment. The consistency checking system 1 includes an electronic medical record system 10, a data management system 20, and a medical information processing apparatus 30. Moreover, the systems and apparatuses included in the consistency checking system 1 are communicably connected to each other via a network 40. The configuration illustrated in FIG. 1 is merely an example, and the number of systems and apparatuses may be optionally changed.

Furthermore, apparatuses not illustrated in FIG. 1 may also be connected to the network 40.

Health care professionals have been entering the progress of medical treatment on paper medical records. In recent years, paper medical records have been converted into electronic medical records to be stored as data. For example, health care professionals convert paper medical records into electronic medical records, by entering what is written on the paper medical record into the electronic medical records.

In such a case, errors may be input to the electronic medical records due to input mistakes. Then, if a health care professional gives medical treatment on the basis of the error, a medical accident may occur. Moreover, in the electronic medical records, to support the health care professional in future medical treatments, the progress of medical treatment may be analyzed statistically. Thus, authenticity indicating that the electronic medical record does not contain errors is required.

Therefore, there are measures for extracting errors from the electronic medical records using natural language processing. However, although the natural language processing can be used to determine whether the context of the electronic medical records is correct, it is difficult to determine whether the input content is correct. For example, if the term “patient's body temperature is 39.0 degrees Celsius” is input into the electronic medical records, it is difficult to determine whether the patient's body temperature was actually 39.0 degrees Celsius, using natural language processing. Moreover, it is time-consuming to manually check whether the electronic medical records contain errors.

Therefore, the consistency checking system 1 determines whether the medical treatment information of a patient is consistent with the medical information of the same patient. For example, the consistency checking system 1 compares between the items in the electronic medical records to determine whether there is consistency.

In this example, the electronic medical records are input using a Subjective Objective Assessment Plan (SOAP) format. The SOAP format is a description format that describes the patient's subjective information as claimed by the patient, the objective information as viewed by the health care professional, the assessment on the basis of the subjective information and objective information, and the plans for diagnosis, treatment, and education on the basis of the assessment, in each item. Thus, for example, if information inconsistent with the subjective information or objective information is in the assessment item, the inconsistent information is likely to be an error.

For example, the consistency checking system 1 determines whether the body temperature of a patient entered into the electronic medical records is consistent with the body temperature of the patient in the database. Then, if the body temperature of the patient entered into the electronic medical records is inconsistent with the body temperature of the patient in the database, the body temperature information in the electronic medical records is likely to be an error. In this manner, the consistency checking system 1 detects an error in the medical treatment information, by determining whether the medical treatment information is consistent with the medical information.

The consistency checking system 1 will be described in detail.

For example, the electronic medical record system 10 is implemented by a computer apparatus such as a server and a workstation. The electronic medical record system 10 stores medical treatment information that is the medical treatment record of the patient. For example, the medical treatment information is electronic medical records in which the SOAP format is used to input character strings. That is, the medical treatment information is medical information used for medical treatment.

Moreover, the electronic medical record system 10 includes a database of patient medical information. For example, as medical information, the database includes test results obtained from various tests performed on the patient. For example, in the database, the date and time of the test is associated with the test results for each patient. For example, as the test results, the database includes medical information such as body temperature, blood pressure, pulse rate, respiratory rate, blood test results, urine test results, and imaging diagnostic results.

For example, the data management system 20 is implemented by a computer apparatus such as a server and a workstation. The data management system 20 is a system that manages medical information of patients. For example, the data management system 20 is a system that stores medical information of patients that is not stored in the electronic medical record system 10. For example, the data management system 20 includes a database in which the date and time of the test is associated with the test results for each patient. The data management system 20 may also include other medical information in addition to the test results.

For example, the medical information processing apparatus 30 is implemented by a computer apparatus such as a server and a workstation. The medical information processing apparatus 30 determines whether the medical treatment information is consistent with the medical information. For example, the medical information processing apparatus 30 obtains medical treatment information from the electronic medical record system 10. Moreover, the medical information processing apparatus 30 determines whether the content indicated by the character string obtained from the medical treatment information is consistent with the content indicated by the character string in the other item of the medical treatment information or the content indicated by the character string of the medical information in the database, by comparing therebetween.

The medical information processing apparatus 30 will be described in detail.

The medical information processing apparatus 30 includes network (NW) interface circuitry 310, input interface circuitry 320, a display 330, a storage 340, and processing circuitry 350.

The NW interface circuitry 310 is connected to the processing circuitry 350, and controls the transmission and communication of various types of data performed between the apparatuses connected via the network 40. For example, the NW interface circuitry 310 is implemented by a network card, a network adapter, a network interface controller (NIC), and the like.

The input interface circuitry 320 is connected to the processing circuitry 350, converts an input operation received from the operator (health care professional) into an electrical signal, and outputs the electrical signal to the processing circuitry 350. Specifically, the input interface circuitry 320 converts an input operation received from the operator into an electrical signal, and outputs the electrical signal to the processing circuitry 350. For example, the input interface circuitry 320 is implemented by a trackball, a switch button, a mouse, a keyboard, a touch pad with which input operations are performed by touching an operation surface, a touch screen in which a display screen and a touch pad are integrated, a non-contact input circuit using an optical sensor, a voice input circuit, and the like. In the present specification, the input interface circuitry 320 is not limited to one having physical operation parts such as a mouse and a keyboard. For example, an electrical signal processing circuit that receives an electrical signal corresponding to an input operation from an external input apparatus, which is provided separately from the apparatus, and that outputs the electrical signal to a control circuit, is also an example of the input interface circuitry 320.

The display 330 is connected to the processing circuitry 350 and displays various types of information and image data output from the processing circuitry 350. For example, the display 330 is implemented by a liquid crystal display, a cathode ray tube (CRT) display, an organic EL display, a plasma display, a touch panel, and the like.

The storage 340 is connected to the processing circuitry 350, and stores various types of data. Moreover, the storage 340 stores various computer programs for implementing various functions that are read and executed by the processing circuitry 350. For example, the storage 340 is implemented by a semiconductor memory device such as a random access memory (RAM) and flash memory, a hard disk, an optical disc, and the like.

The processing circuitry 350 controls the entire operation of the medical information processing apparatus 30. For example, the processing circuitry 350 includes a condition setting function 351, a processing target acquisition function 352, a comparison target acquisition function 353, a consistency determination function 354, and a determination result output function 355. In the embodiment, each processing function performed by the condition setting function 351, the processing target acquisition function 352, the comparison target acquisition function 353, the consistency determination function 354, and the determination result output function 355 serving as components, is stored in the storage 340 in the form of a computer executable program. The processing circuitry 350 is a processor that reads and executes a computer program from the storage 340, and implements the function corresponding to each computer program. In other words, the processing circuitry 350 that has read out computer programs has the functions illustrated in the processing circuitry 350 in FIG. 1.

In FIG. 1, the condition setting function 351, the processing target acquisition function 352, the comparison target acquisition function 353, the consistency determination function 354, and the determination result output function 355 are implemented by a single processor. However, the processing circuitry 350 can also be configured by combining a plurality of independent processors, and each processor can execute a computer program to implement the function. Moreover, in FIG. 1, a single storage circuit such as the storage 340 stores a computer program corresponding to each processing function. However, a plurality of storage circuits may be distributed and arranged, and the processing circuitry 350 may read the corresponding computer program from the storage circuit independently.

For example, the term “processor” used in the above description refers to a central processing unit (CPU), a graphical processing unit (GPU), or a circuit such as an application specific integrated circuit (ASIC) and a programmable logic device (for example, a simple programmable logic device (SPLD), a complex programmable logic device (CPLD), and a field programmable gate array (FPGA)). The processor implements functions by reading and executing a computer program stored in the storage 340. Instead of storing a computer program in the storage 340, a computer program may also be directly embedded in a circuit of the processor. In this case, the processor implements the function by reading and executing a computer program embedded in the circuit.

The condition setting function 351 sets conditions to be specified in the medical treatment information the consistency of which is to be determined. The condition setting function 351 is an example of a setting unit. For example, the condition setting function 351 sets conditions such as the period during which the character string is input to the medical treatment information, the facility where the medical treatment information is generated, the department where the character string is input to the medical treatment information, the patient of the medical treatment information, the type of the medical treatment information, and the health care professional who has input the medical treatment information. Moreover, the condition setting function 351 may set multiple conditions in addition to one condition.

The processing target acquisition function 352 acquires a first character string from the medical treatment information. The processing target acquisition function 352 is an example of a first acquisition unit. In this example, the first character string is a character string the consistency of which is to be determined. For example, the first character string is one or more characters including numbers and symbols.

More specifically, the processing target acquisition function 352 acquires one or more pieces of medical treatment information that satisfy the conditions set by the condition setting function 351, from the electronic medical record system 10. Moreover, the processing target acquisition function 352 acquires the first character string from each of one or more pieces of the medical treatment information that satisfy the conditions set by the condition setting function 351. Furthermore, the processing target acquisition function 352 may acquire the first character string in word units, may acquire the first character string in sentence units, or may acquire the first character string in item units.

Still furthermore, when the medical treatment information is electronic medical records, the processing target acquisition function 352 acquires the first character string from the first item of the medical treatment information. For example, the first item is an item for the assessment on the basis of subjective information and objective information, or the plans for diagnosis, treatment, or education on the basis of the assessment.

The comparison target acquisition function 353 acquires one or more second character strings estimated from the first character string, from at least one of the medical treatment information and one or more pieces of medical information that is different from the medical treatment information. The comparison target acquisition function 353 is an example of a second acquisition unit. In this example, the second character string is a character string to be compared with the first character string, in determining the consistency. For example, the second character string is one or more characters including numbers and symbols. Moreover, to determine the consistency, the second character string must be a character string indicating the same content as that of the first character string. That is, the comparison target acquisition function 353 acquires one or more second character strings that are estimated to have the same content as that of the first character string.

For example, the comparison target acquisition function 353 acquires the second character string from the medical information such as the subjective information and objective information contained in the medical treatment information. Moreover, the comparison target acquisition function 353 acquires the second character string from the medical information in the database.

More specifically, the comparison target acquisition function 353 specifies the medical information to be compared, on the basis of the content of the first character string. For example, the comparison target acquisition function 353 identifies the content indicated by the first character string, by performing natural language processing on the first character string. Moreover, the comparison target acquisition function 353 specifies the medical information corresponding to the content indicated by the first character string, on the basis of the list information indicating the content of each medical information. Furthermore, the comparison target acquisition function 353 may specify the database of the medical information in the electronic medical record system 10 and the database of the medical information in the data management system 20.

Still furthermore, the comparison target acquisition function 353 acquires one or more second character strings from the medical information specified on the basis of the content of the first character string obtained by the processing target acquisition function 352. For example, the comparison target acquisition function 353 acquires one or more second character strings indicating the same content as that of the first character string from the specified medical information, on the basis of the item list information indicating the content of each item of the medical information. Still furthermore, the comparison target acquisition function 353 may acquire one or more second character strings from the medical information contained in the data base specified on the basis of the content of the first character string obtained by the processing target acquisition function 352.

Still furthermore, when the content indicated by the first character string obtained by the processing target acquisition function 352 contains the period content indicating the period, the comparison target acquisition function 353 acquires the second character string of the period that matches with the period content. For example, when the first character string obtained by the processing target acquisition function 352 contains a character string of “than the body temperature measured before last”, the comparison target acquisition function 353 acquires the second character string indicating the body temperature measured before last from the database. In addition to the method described above, the comparison target acquisition function 353 may also acquire the second character string from the medical information to be compared by using a learned model, or may acquire the second character string from the medical information using other methods.

Moreover, if the first character string is obtained from the medical treatment information by the processing target acquisition function 352, the comparison target acquisition function 353 may acquire the second character string from the subjective information or objective information of the medical treatment information from which the first character string is obtained.

Furthermore, when the medical treatment information is electronic medical records, the comparison target acquisition function 353 acquires the second character string from the second item of the medical treatment information. For example, the second item is an item for the subjective information or objective information.

The consistency determination function 354 determines whether the content indicated by the first character string is consistent with the content indicated by the second character string obtained by the comparison target acquisition function 353. The consistency determination function 354 is an example of a determination unit. More specifically, the consistency determination function 354 uses natural language processing to obtain the content indicated by each of the first character string and the second character string. Then, the consistency determination function 354 determines whether the content indicated by the first character string is consistent with the content indicated by the second character string, by comparing therebetween. Moreover, when multiple second character strings are obtained by the comparison target acquisition function 353, the consistency determination function 354 determines whether the content indicated by the first character string is consistent with the content indicated by the second character string, for each of the second character strings.

When the medical treatment information is electronic medical records, the consistency determination function 354 determines whether the content indicated by the first character string obtained from the first item is consistent with the content indicated by the second character string obtained from the second item. That is, the consistency determination function 354 determines whether the content indicated by the first character string obtained from the assessment or plans in the electronic medical records is consistent with the content indicated by the second character string obtained from the subjective information or objective information in the electronic medical records. Moreover, even when the medical treatment information is electronic medical records, when multiple second character strings are obtained by the comparison target acquisition function 353, the consistency determination function 354 determines whether the content indicated by the first character string is consistent with the content indicated by the second character string, for each of the second character strings.

When the consistency determination function 354 determines that the content indicated by the first character string is inconsistent with the content indicated by the second character string, the determination result output function 355 outputs inconsistency specific information for specifying the location of the first character string. The determination result output function 355 is an example of an output unit. The inconsistency specific information is specific information for specifying the location of the inconsistent first character string. For example, the inconsistency specific information is a patient code for specifying the patient of the medical treatment information including the first character string, an item code for specifying the item including the first character string, and the like. Moreover, the inconsistency specific information may be information indicating the storage destination where the medical information containing the first character string is stored, or may be other information.

Next, a consistency determination process executed by the medical information processing apparatus 30 will be described.

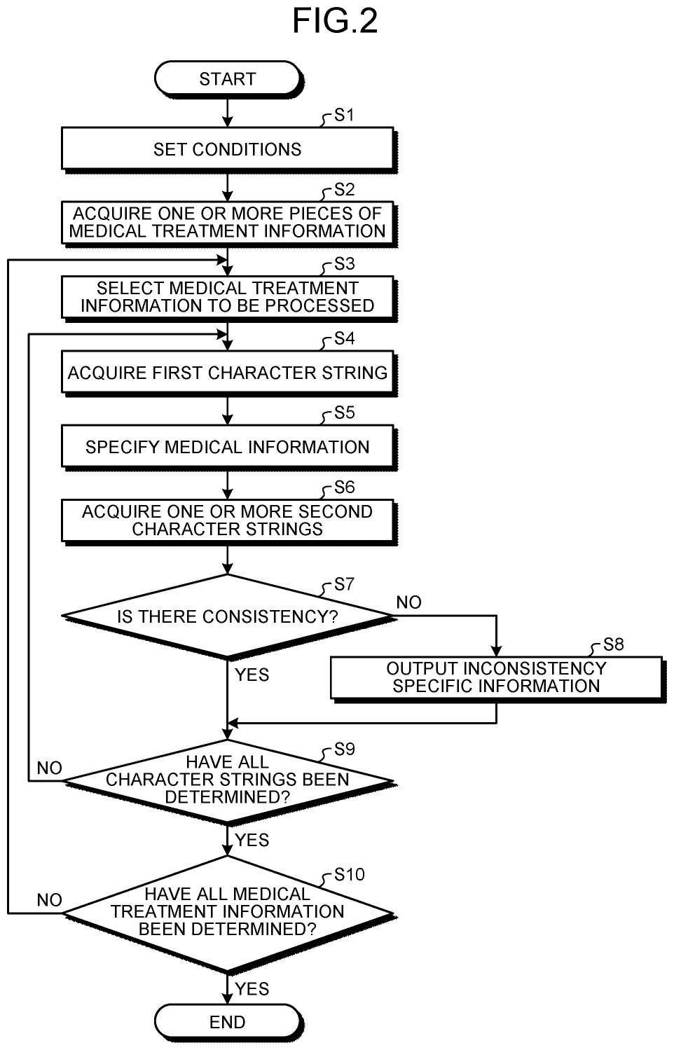

FIG. 2 is a flowchart illustrating an example of a consistency determination process executed by the medical information processing apparatus 30 according to the first embodiment.

The condition setting function 351 sets conditions to be specified in the medical treatment information (step S1).

The processing target acquisition function 352 acquires one or more pieces of medical treatment information that satisfy the conditions set by the condition setting function 351 (step S2).

The processing target acquisition function 352 selects one medical treatment information the consistency of which is to be determined, from one or more pieces of the obtained medical treatment information (step S3).

The processing target acquisition function 352 acquires the first character string the consistency of which is to be determined, from the selected medical treatment information (step S4).

The comparison target acquisition function 353 specifies the medical information to be compared, on the basis of the first character string obtained by the processing target acquisition function 352 (step S5).

The comparison target acquisition function 353 acquires one or more second character strings to be compared with the first character string, from the specified medical information (step S6).

The consistency determination function 354 determines whether the content indicated by the first character string is consistent with the content indicated by the second character string (step S7). When multiple second character strings are obtained, the consistency determination function 354 determines whether the content indicated by the first character string is consistent with the content indicated by the second character string, for each of the second character strings. If there is inconsistency (No at step S7), the determination result output function 355 outputs inconsistency specific information for specifying the location of the first character string (step S8).

If there is consistency (Yes at step S7), the determination result output function 355 determines whether all the first character strings have been determined, among the first character strings to be determined contained in the medical treatment information (step S9). If all the first character strings have not been determined (No at step S9), the processing target acquisition function 352 acquires the first character string that has not yet been determined at step S4.

If all the first character strings have been determined (Yes at step S9), the consistency determination function 354 determines whether all the medical treatment information obtained by the processing target acquisition function 352 have been determined (step S10). If all the medical treatment information have not been determined (No at step S10), the processing target acquisition function 352 selects the medical treatment information that has not yet been determined at step S3.

If all the medical treatment information have been determined (Yes at step S10), the medical information processing apparatus 30 finishes the consistency determination process.

As described above, the medical information processing apparatus 30 according to the first embodiment obtains the first character string from the medical treatment information. Moreover, the medical information processing apparatus 30 obtains one or more second character strings from at least one of the medical treatment information and the medical information. Furthermore, the medical information processing apparatus 30 determines whether the content indicated by the first character string is consistent with the content indicated by the second character string. Then, even if the content indicated by the first character string is determined to be inconsistent with the content indicated by the second character string, the medical information processing apparatus 30 outputs inconsistency specific information for specifying the location of the first character string. The operator can check whether the first character string at the location specified by the inconsistency specific information is actually inconsistent. Then, if the first character string is inconsistent, the operator can correct the medical treatment information. Hence, the medical information processing apparatus 30 according to the first embodiment can support the health care professional in reducing errors in the medical treatment information.

### Second Embodiment

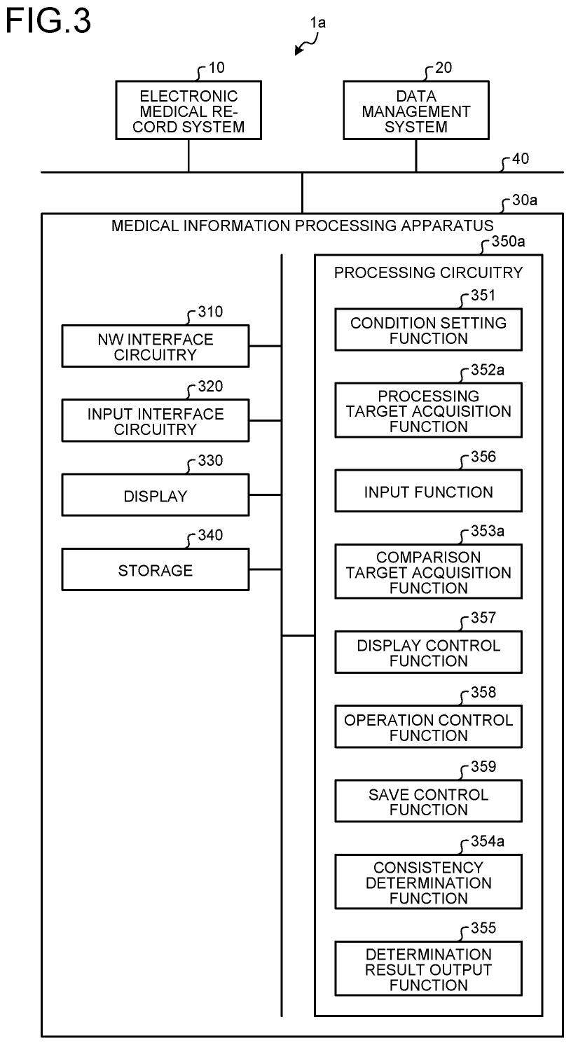

FIG. 3 is a block diagram illustrating an example of a configuration of a consistency checking system 1a according to a second embodiment.

When the medical treatment information is input, the consistency checking system 1a displays input candidates estimated from the input content. Then, when an input candidate is selected, the consistency checking system 1a inputs the selected input candidate. In this manner, the consistency checking system 1a reduces errors in the medical treatment information, by presenting input candidates and reducing input mistakes.

A processing circuitry 350a includes an input function 356, a display control function 357, an operation control function 358, and a save control function 359.

The input function 356 receives an input of the first character string for the medical treatment information. The input function 356 is an example of an input unit. For example, when the medical treatment information is electronic medical records, the input function 356 receives an input of the first character string to the first item of the medical treatment information. That is, when the medical treatment information is electronic medical records, the input function 356 receives an input of the first character string for the assessment on the basis of subjective information and objective information, or the plans for diagnosis, treatment, or education on the basis of the assessment.

A comparison target acquisition function 353a acquires one or more second character strings estimated from the first character string, from the currently input first character string that is being received by the input function 356. That is, the comparison target acquisition function 353a acquires one or more second character strings estimated from the first character string, from at least one of the medical treatment information and one or more pieces of medical information that is different from the medical treatment information.

More specifically, the comparison target acquisition function 353a specifies the medical information to be compared, on the basis of the first character string input by the input function 356. For example, the comparison target acquisition function 353a acquires one or more second character strings to be compared with the first character string that are input by the input function 356, by the same process as that of the comparison target acquisition function 353 according to the first embodiment.

For example, the comparison target acquisition function 353a acquires one or more second character strings from one or more pieces of medical information that is different from the medical treatment information, on the basis of the content of the first character string input by the input function 356. Moreover, when the medical treatment information is electronic medical records, the comparison target acquisition function 353a acquires the second character string from the second item that is different from the first item of the medical treatment information. That is, when the medical treatment information is electronic medical records, the comparison target acquisition function 353a acquires the second character string from the subjective information or objective information.

A consistency determination function 354a determines whether the content indicated by the first character string is consistent with the content indicated by the second character string to be compared that is obtained by the comparison target acquisition function 353a. More specifically, the consistency determination function 354a uses natural language processing to obtain the content indicated by each of the first character string and the second character string. Then, the consistency determination function 354a determines whether the content indicated by the first character string is consistent with the content indicated by the second character string, by comparing therebetween. Moreover, when multiple second character strings are obtained by the comparison target acquisition function 353a, the consistency determination function 354a determines whether the content indicated by the first character string is consistent with the content indicated by the second character string, for each of the second character strings.

The display control function 357 displays an input candidate image G15 including the input candidates on the basis of the second character string determined to be consistent by the consistency determination function 354a. Moreover, if one or more second character strings are obtained by the comparison target acquisition function 353a, the display control function 357 displays the input candidate image G15 including input candidates on the basis of each of one or more of the second character strings. The display control function 357 is an example of a display control unit.

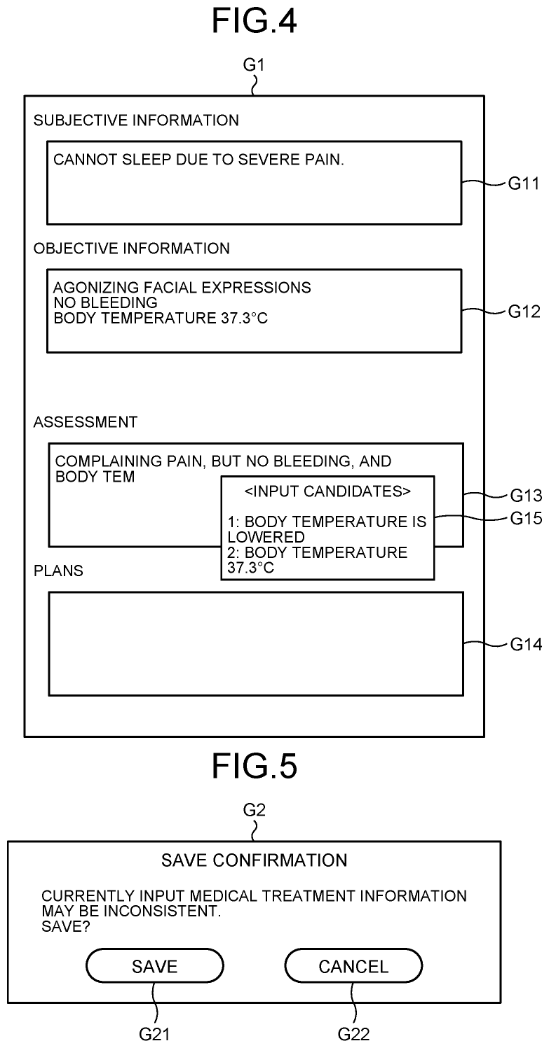

FIG. 4 is a diagram illustrating an example of the input candidate image G15. The input candidate image G15 is an image illustrating input candidates. The input candidates are character strings to be input. Moreover, each of the input candidates may be the second character string or a character string the content of which is estimated from the second character string. Furthermore, the display control function 357 may display a character string the content of which is estimated from the second character string determined to be consistent by the consistency determination function 354a, on the display candidate image G15.

As illustrated in FIG. 4, the display control function 357 displays the input candidate image G15 on an input image G1 of the medical treatment information in an overlapping manner. The input image G1 includes a first input field G11 for subjective information, a second input field G12 for objective information, a third input field G13 for assessment, and a fourth input field G14 for plans, of the electronic medical records.

The input candidate image G15 illustrated in FIG. 4 illustrates a state in which the first character string is input to the third input field G13 for assessment by the input function 356, in the input image G1 of the medical treatment information. In the input image G1 illustrated in FIG. 4, the comparison target acquisition function 353a acquires one or more second character strings from the first input field G11 for subjective information or from the second input field G12 for objective information, on the basis of the first character string input to the third input field G13. The consistency determination function 354a determines whether the content indicated by the first character string is consistent with the content indicated by the second character string. Then, the display control function 357 displays the input candidate image G15 illustrated in FIG. 4, on the basis of the second character string determined to be consistent by the consistency determination function 354a.

Moreover, the display control function 357 displays a save confirmation image G2. FIG. 5 is a diagram illustrating an example of the save confirmation image G2. The save confirmation image G2 is an image that, upon receiving an operation of saving the currently input medical treatment information, asks the user whether to save the currently input medical treatment information, although the currently input medical treatment information may contain an inconsistent first character string. The save confirmation image G2 includes a save button G21 and a cancel button G22. The save button G21 is a button for receiving an operation for saving the medical treatment information. The cancel button G22 is a button for receiving an operation of canceling the medical treatment information.

The operation control function 358 receives various operations. For example, the operation control function 358 receives an operation for selecting an input candidate to be input, from one or more input candidates included in the input candidate image G15. Moreover, the operation control function 358 receives an operation of saving the medical treatment information input to the input image G1. For example, the operation control function 358 receives an operation of pressing the save button G21 or cancel button G22 in the save confirmation image G2.

The save control function 359 stores medical treatment information in the storage media of the electronic medical record system 10. For example, when the saving operation is received by the operation control function 358, the save control function 359 stores medical treatment information. Moreover, when the save confirmation image G2 is displayed and when the save button G21 on the save confirmation image G2 is pressed, the save control function 359 stores the medical treatment information.

When an operation of saving the medical treatment information is received by the operation control function 358, a processing target acquisition function 352a, the comparison target acquisition function 353a, and the consistency determination function 354a determine whether the medical treatment information to be saved contains an inconsistent first character string. Then, when an inconsistent first character string is contained, the determination result output function 355 outputs inconsistency specific information for specifying the inconsistent first character string. Moreover, the display control function 357 displays a save confirmation image G2.

Next, an input support process executed by a medical information processing apparatus 30a will be described.

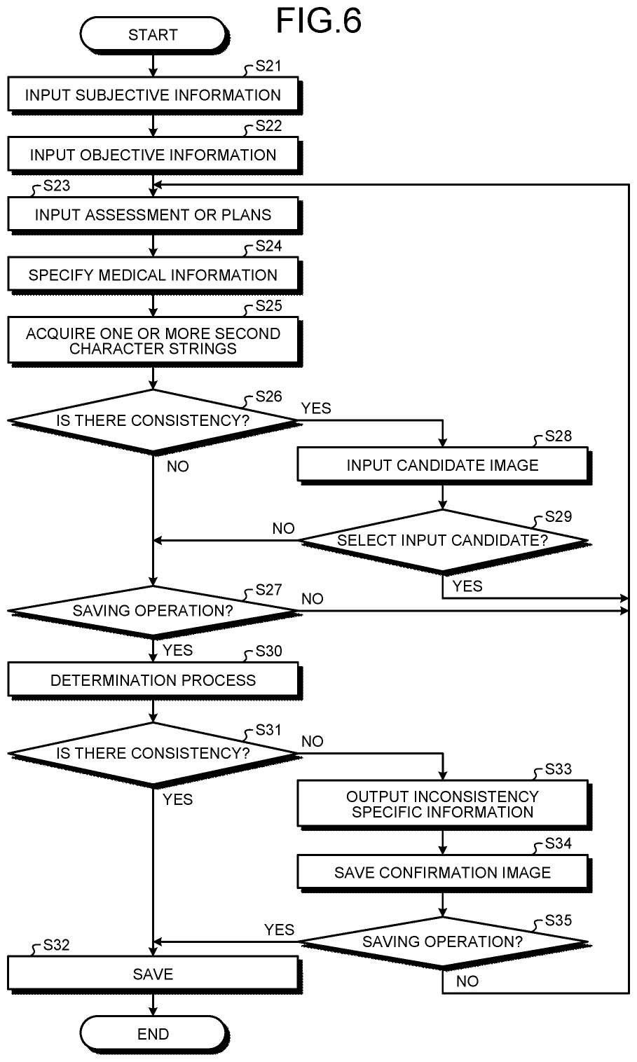

FIG. 6 is a flowchart illustrating an example of an input support process executed by the medical information processing apparatus 30a according to the second embodiment.

The input function 356 receives an input of a character string to the first input field G11 for subjective information in the input image G1 (step S21).

The input function 356 receives an input of a character string to the second input field G12 for objective information in the input image G1 (step S22).

The input function 356 receives an input of the first character string to the third input field G13 for assessment or the fourth input field G14 for plans in the input image G1 (step S23).

The comparison target acquisition function 353a specifies the medical information to be compared, on the basis of the first character string input by the input function 356 (step S24).

The comparison target acquisition function 353a acquires one or more second character strings to be compared with the first character string, from the specified medical information (step S25).

The consistency determination function 354a determines whether the content indicated by the first character string is consistent with the content indicated by the second character string (step S26). When multiple second character strings are obtained, the consistency determination function 354a determines whether the content indicated by the first character string is consistent with the content indicated by the second character string, for each of the second character strings. If there is inconsistency (No at step S26), the medical information processing apparatus 30a proceeds to step S27.

If there is consistency (Yes at step S26), the display control function 357 displays the input candidate image G15 on the basis of one or more second character strings that are consistent (step S28).

The operation control function 358 determines whether an operation of selecting an input candidate included in the input candidate image G15 is received (step S29). If the operation of selecting an input candidate is received (Yes at step S29), the input function 356 receives the character string of the input candidate selected at step S23. If the operation of selecting an input candidate is not received (No at step S29), the medical information processing apparatus 30a proceeds to step S27.

The operation control function 358 determines whether an operation of saving the medical treatment information input to the input image G1 is received (step S27). If the saving operation is not received (No at step S27), the input function 356 proceeds to step S23 to receive the input of the first character string.

If the saving operation is received (Yes at step S27), the medical information processing apparatus 30a performs a process of determining whether the medical treatment information input to the input image G1 contains an inconsistent character string (step S30). The medical information processing apparatus 30a performs the processes from step S4 to step S9 of the flowchart illustrated in FIG. 2 for the medical treatment information input to the input image G1.

The consistency determination function 354a determines whether the medical treatment information contains an inconsistent first character string, on the basis of the processing results (step S31).

If the medical treatment information does not contain the inconsistent first character string (Yes at step S31), the save control function 359 saves the medical treatment information input to the input image G1 (step S32).

If the medical treatment information contains the inconsistent first character string (No at step S31), the determination result output function 355 outputs inconsistency specific information for specifying the inconsistent first character string (step S33). The inconsistency specific information may also be information that highlights the inconsistent first character string in the medical treatment information.

The display control function 357 displays the save confirmation image G2 to ask the user whether to save the medical treatment information, although the medical treatment information may contain an inconsistent first character string (step S34).

The operation control function 358 determines whether an operation of saving the medical treatment information is received (step S35). That is, the operation control function 358 determines whether an operation of pressing the save button G21 on the save confirmation image G2 is received.

When an operation of pressing the cancel button G22 on the save confirmation image G2 is received (No at step S35), the input function 356 receives an input of correcting the inconsistent first character string at step S23.

When the operation of pressing the save button G21 on the save confirmation image G2 is received (Yes at step S35), the save control function 359 saves the medical treatment information input to the input image G1 at step S32.

Accordingly, the medical information processing apparatus 30a finishes the input support process.

The medical information processing apparatus 30a according to the second embodiment receives an input of the first character string for the medical treatment information. Moreover, the medical information processing apparatus 30a obtains one or more second character strings from at least one of the medical treatment information and medical information. Furthermore, the medical information processing apparatus 30a determines whether the content indicated by the first character string is consistent with the content indicated by the second character string. Then, the medical information processing apparatus 30a displays input candidates on the basis of the second character string determined to be consistent. In this manner, the operator can input a character string by selecting an input candidate. Moreover, the operator is less likely to input an incorrect character string, because the operator selects an input candidate. Hence, the medical information processing apparatus 30a according to the second embodiment can support the health care professional in reducing errors in the medical treatment information.

### First Modification

The medical information processing apparatuses 30 and 30a include the condition setting function 351, the processing target acquisition functions 352 and 352a, the input function 356, the comparison target acquisition functions 353 and 353a, the display control function 357, the operation control function 358, the save control function 359, the consistency determination functions 354 and 354a, and the determination result output function 355. However, an apparatus other than the medical information processing apparatuses 30 and 30a may also include all or part of the functional units. For example, the electronic medical record system 10 or the data management system 20 may include all or part of the functional units. An apparatus or a system not illustrated in FIG. 1 may also include all or part of the functional units.

### Second Modification

The medical information processing apparatuses 30 and 30a implement the condition setting function 351, the processing target acquisition functions 352 and 352a, the input function 356, the comparison target acquisition functions 353 and 353a, the display control function 357, the operation control function 358, the save control function 359, the consistency determination functions 354 and 354a, and the determination result output function 355, by executing a computer program stored in the storage 340. However, the medical information processing apparatuses 30 and 30a may also implement all or part of the condition setting function 351, the processing target acquisition functions 352 and 352a, the input function 356, the comparison target acquisition functions 353 and 353a, the display control function 357, the operation control function 358, the save control function 359, the consistency determination functions 354 and 354a, and the determination result output function 355, using hardware such as a semiconductor circuit.

According to at least one of the embodiments described above and the like, it is possible to support the health care professional in reducing errors in the medical treatment information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus, comprising:
processing circuitry configured to
acquire a first character string from medical treatment information,
acquire one or more second character strings estimated from the first character string, from at least one of the medical treatment information and one or more pieces of medical information that is different from the medical treatment information,
determine whether a content indicated by the first character string is consistent with a content indicated by the second character string, and
output specific information for specifying a location of the first character string, if the content indicated by the first character string is inconsistent with the content indicated by the second character string.

2. The medical information processing apparatus according to claim 1, wherein the processing circuitry is configured to
sets a condition to be specified in the medical treatment information, and
acquires the first character string from each of one or more pieces of the medical treatment information that satisfy the set condition.

3. The medical information processing apparatus according to claim 1, wherein the processing circuitry is configured to acquires one or more of the second character strings from the medical information specified based on the content of the first character string.

4. The medical information processing apparatus according to claim 1, wherein the processing circuitry is configured to acquires one or more of the second character strings from the medical information contained in a data base specified based on the content of the first character string.

5. The medical information processing apparatus according to claim 1, wherein the processing circuitry is configured to
acquires the first character string from a first item of the medical treatment information,
acquires the second character string from a second item that is different from the first item of the medical treatment information, and
determines whether the content indicated by the first character string obtained from the first item is consistent with the content indicated by the second character string obtained from the second item.

6. The medical information processing apparatus according to claim 1, wherein the processing circuitry is configured to displays an input candidate based on the second character string determined to be consistent.

7. The medical information processing apparatus according to claim 6, wherein the processing circuitry is configured to
acquires one or more of the second character strings from one or more pieces of the medical information that is different from the medical treatment information, based on the content of the first character string, and
displays each of the input candidate based on each of one or more of the second character strings.

8. The medical information processing apparatus according to claim 6, wherein the processing circuitry is configured to
receives an input of the first character string for a first item of the medical treatment information,
acquires the second character string from a second item that is different from the first item of the medical treatment information, and
displays each of the input candidate based on each of one or more of the second character strings.

9. A consistency determination method, comprising:
acquiring a first character string from medical treatment information;
acquiring one or more second character strings estimated from the first character string, from at least one of the medical treatment information and one or more pieces of medical information that is different from the medical treatment information;
determining whether a content indicated by the first character string is consistent with a content indicated by the second character string; and
outputting specific information for specifying a location of the first character string, if the content indicated by the first character string is inconsistent with the content indicated by the second character string.

10. The consistency determination method according to claim 9, further comprising displaying an input candidate based on the second character string determined to be consistent.
